(19) United States
(12) Patent Application Publication
(10) Pub. No.: US 2023/0097381 A1
(43) Pub. Date: Mar. 30, 2023

(54) COMPOSITION, KIT AND METHOD FOR DIAGNOSIS AND TREATMENT OF PROSTATE CANCER

(71) Applicant: RADIOMEDIX, INC., Houston, TX (US)

(72) Inventors: Ebrahim Delpassand, Houston, TX (US); Izabela Tworowska, Houston, TX (US)

(73) Assignee: RADIOMEDIX, INC., Houston, TX (US)

(21) Appl. No.: 17/996,958

(22) PCT Filed: Apr. 26, 2021
(86) PCT No.: PCT/US2021/029124

§ 371 Date: Oct. 24, 2022

Related U.S. Application Data
(60) Provisional application No. 63/015,182, filed on Apr. 24, 2020.

(51) Int. Cl. A61K 51/08; A61K 51/04; A61P 35/00; A61P 15/00

(52) U.S. Cl. CPC A61K 51/088; A61K 51/0482; A61P 35/00; A61P 15/00

(57) ABSTRACT

Disclosed herein are compositions, kits and methods for treating and detecting cancer, and more particularly radiolabeled conjugates used for targeted radiotherapy of cancer patients.

### RELATED CASE

This application claims priority to U.S. Provisional Application No. 63/015,182 filed on Apr. 24, 2020, which is incorporated herein by reference in its entirety to the full extent permitted by law.

### BACKGROUND

The present disclosure relates generally to cancer treatment. More particularly, the present disclosure relates to targeted radiotherapy of cancer patients using radiolabeled conjugates.

Various medications have been developed for the treatment of cancer cells. In order to specifically target the cancer cells, targeting compositions have been developed to treat to the cancer cells without affecting healthy cells which may be near the cancer cells. To target the cancer cells, the targeting compositions are provided with chemicals which are designed to bind specifically to portions of the cancer cells. Such compositions may be overexpressed in cancer cells compared to healthy cells. These compositions are also designed to bind to and damage the cancer cells without damaging other cells in the patient.

Examples of conjugates used in cancer treatment are provided in US Patent/Application Nos. 2016/0143926, 2015/0196673, 2014/0228551, 9408928, 9217009, 8858916, 7202330, 6225284, 6683162, 6358491, and WO2014052471, the entire contents of which are hereby incorporated by reference herein. Examples of tumor targeting compositions are provided in US Patent/Application Nos. US2007/0025910, and U.S. Pat. No. 5,804,157, the entire contents of which are hereby incorporated by reference herein.

Additional information concerning cancer treatment is provided below.

### BRIEF DESCRIPTION OF THE DRAWINGS

The present disclosure is best understood from the following detailed description when read with the accompanying Figures. It is emphasized that, in accordance with the standard practice in the industry, various features are not drawn to scale. In fact, the dimensions of the various features may be arbitrarily increased or reduced for clarity of discussion.

FIG. 1 depicts the microPET imaging studies of ⁶⁴Cu-DOTAM-PSMA (injected dose 45 uCi) in LNCap (left flank) and 22Rv1(right flank) xenografts generated in the Athymic Nude Mice. Images were acquired 1 h post-injection. The photos of mice (on left) are showing the actual size of the implanted tumors.

FIG. 2 depicts microPET imaging studies of ⁶⁴Cu-DOTAM-PSMA in LNCap (left flank) and 22Rv1(right flank) xenograft mice done at 2 h post-injection; a) the reconstructed fused PET/CT scan; b) coronal view; c) axial view. The agent is retained in both LNCap and 22Rv1-derived tumors, according to one or more examples of the disclosure.

FIG. 3 depicts microPET imaging studies of ⁶⁴Cu-DOTAM-PSMA (62.3 uCi) in LNCap (left flank, volume 500 mm³) and 22Rv1 (right flank, volume 192 mm³) xenografts mice done at 4 h post-injection; a) the reconstructed PET/CT fused scans; b) the sagittal view; c) coronal view; d) axial view. The agent is retained in both LNCap and 22Rv1 tumors as well as non-target organ, liver, according to one or more examples of the disclosure.

FIG. 4 shows graphs plotting the time-dependent changes in distribution of ⁶⁴Cu-DOTAM-PSMA in 22RV1 tumor and normal organs (liver, kidneys, muscle and salivary glands), according to one or more examples of the disclosure.

FIG. 5A depicts microPET imaging studies of ⁶⁴Cu-DOTAM-PSMA in LNCap (left flank) and 22Rv1(right flank) xenografts generated in the athymic nude mice. The scans were acquired at 1 h post-injection. The tumors volumes were below 150 mm³.

FIG. 5B are photos of mice showing size of the implanted tumors, according to one or more examples of the disclosure.

FIG. 6 depicts microPET imaging studies of ⁶⁴Cu-DOTAM-PSMA in LNCap xenografts generated in NOG mice; Studies were done at 1 h (A) and 24 h (B) post-injection, according to one or more examples of the disclosure.

FIG. 7 shows graphs plotting the biodistribution studies of ⁶⁴Cu-DOTAM-PSMA in athymic nude mice done at 1 h, 2 h and 24 h post-injection. The liver and kidneys are the off-target organs showing the highest accumulation of agents, according to one or more examples of the disclosure.

FIG. 8 shows graphs plotting the biodistribution studies of ⁶⁴Cu-DOTAM-PSMA in LNCap and 22RV1 xenografts of R2G2 mice done at 2 h and 24 h post-injection and of NOG mice done at 1 h and 24 h post-injection, according to one or more examples of the disclosure.

FIG. 9 depicts biodistribution results of ²¹²Pb-DOTAM-PSMA administered to PSMA-overexpressing xenografts of athymic nude mice done at 1 h and 3 h post-injection.

FIG. 10 represents the side by side comparison of accumulation of ²¹²Pb-DOTAM-PSMA in LNCAP xenografts at 1 h and 3 h post-injection.

FIG. 11 depicts biodistribution results of ²⁰³Pb-DOTAM-PSMA administered to PSMA-overexpressing xenografts of athymic nude mice done at 1 h post-injection.

FIG. 12 represents biodistribution results of ²⁰³Pb-DOTAM-PSMA administered to PSMA-overexpressing xenografts of athymic nude mice done at 3 h post-injection.

FIG. 13A shows a select radio-HPLC chromatogram of Pb203-RMX-PSMA stored for 1 hour at room temperature. Retention time (Rt) of the radiolabeled product is 14.7 min.

FIG. 13B shows a select radio-HPLC chromatogram of Pb203-RMX-PSMA stored for 48 hours at room temperature. Retention time (Rt) of the radiolabeled product is 14.7 min.

FIG. 13C shows a select radio-HPLC chromatogram of Pb203-RMX-PSMA stored for 72 hours at room temperature. Retention time (Rt) of the radiolabeled product is 14.7 min.

### DETAILED DESCRIPTION

The description that follows includes exemplary apparatus, methods, techniques, and/or instruction sequences that embody techniques of the present subject matter. However, it is understood that the described embodiments may be practiced without these specific details.

Prostate-specific membrane antigen (PSMA) is uniquely overexpressed on the surface of prostate cancer cells as well as in the neovasculature of a variety of solid tumors. As a result, PSMA has attracted attention as a clinical biomarker for detection and management of prostate cancer. Generally, these approaches utilize an antibody specifically targeted at PSMA to direct imaging or therapeutic agents. For example, ProstaScint (Cytogen, Philadelphia, Pa.), which has been approved by the FDA for the detection and imaging of prostate cancer, utilizes an antibody to deliver a chelated radioisotope (Indium-111). However, it is now recognized that the ProstaScint technology is limited to the detection of dead cells and therefore its clinical relevance is questionable.

The success of cancer diagnosis and therapy using antibodies is limited by challenges such as slow elimination of these biomolecules from the blood and poor vascular permeability. In addition, large antibodies bound to cell-surface targets present a barrier for subsequent binding of additional antibodies at neighboring cell-surface sites resulting in a decreased cell-surface labeling.

In addition to serving as a cell-surface target for antibodies delivering diagnostic or therapeutic agents, a largely over-looked and unique property of PSMA is its enzymatic activity. That is, PSMA is capable of recognizing and processing molecules as small as dipeptides. Despite the existence of this property, it has been largely unexplored in terms of the development of novel diagnostic and therapeutic strategies. There are a few recent examples in the literature that have described results in detecting prostate cancer cells using labeled small-molecule inhibitors of PSMA.

In at least one aspect, the disclosure relates to a cancer targeting composition for treatment of cancer cells overexpressing PSMA. The composition comprises a radioisotope, a chelator, and a targeting moiety. In one embodiment, the chelator comprises a nitrogen ring structure, for example, DOTAM.

The chelator (DOTAM) may have the following general formula:

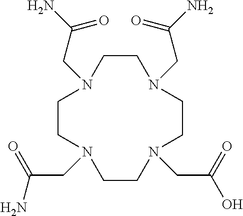

The nitrogen ring structure may comprise a derivative selected from the group consisting of a tetraazacyclododecane derivative, a triazacyclononane derivative, and a tetraazabicyclo[6.6.2] hexadecane derivative. The targeting moiety may comprise a PMSA receptor targeting peptide. The PSMA receptor targeting peptide may be conjugated to the chelator coordinating the radioisotope whereby the cancer cells are targeted for elimination and treated. For example, the chelator DOTAM may be conjugated to the targeting moiety via a covalent bond at its carboxylic acid substituent. The radioisotope may be any radioisotope useful for imaging cancers, including prostate and colorectal cancers, as well as any radioisotope useful for treating cancer, including prostate and colorectal cancers. In some embodiments, the radioisotope may be ⁶⁴Cu, ⁶⁷Cu, ²⁰³Pb, or ²¹²Pb.

A cancer targeting composition for treatment of cancer cells overexpressing PSMA receptors is disclosed herein. The cancer targeting composition includes a radioisotope; a chelator comprising a nitrogen ring structure, the nitrogen ring structure comprising DOTAM, and a targeting moiety comprising a PSMA receptor targeting peptide, with the targeting moiety being conjugated to the chelator coordinating the radioisotope whereby the cancer cells are targeted for elimination and treated; or a product thereof.

In one embodiment, the cancer targeting composition is DOTAM-PSMA having the following general formula:

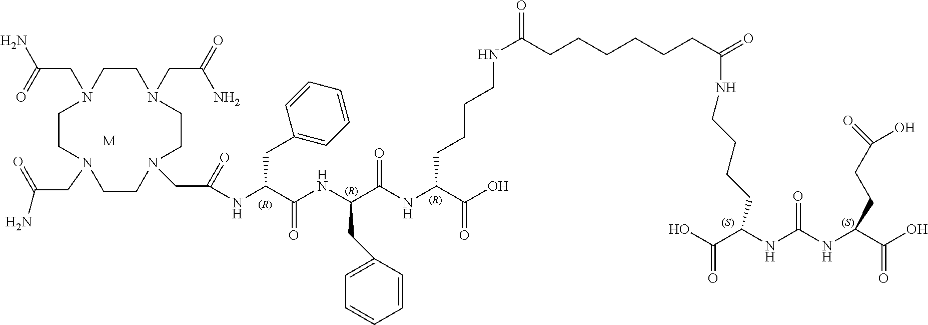

where M is a radioisotope. In one embodiment, the radioisotope is ⁶⁴Cu. In another embodiment, the radioisotope is ⁶⁷Cu. In still another embodiment, the radioisotope is ²⁰³Pb. In yet another embodiment, the radioisotope is ²¹²Pb. The disclosure herein is not limited by the PSMA-targeting moiety in the above structure but may encompass any PSMa-targeting moiety shown to sufficiently bind the PSMA receptors on the surface of cancer cells.

The compounds of the present invention may take the form of salts when appropriately substituted with groups or atoms capable of forming salts. Such groups and atoms are well known to those of ordinary skill in the art of organic chemistry. The term “salts” embraces addition salts of free acids or free bases which are compounds of the invention. The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts.

While the methods and compositions described herein relate to certain cancer treatment, such may also be applicable to cardiovascular disease, infection, diabetes, cancer, and/or other conditions. For cases involving cancer, the cancer may be, for example, a solid tumor derived, for example, either primarily or as a metastatic form, from cancers such as of the liver, prostate, pancreas, head and neck, breast, brain, colon, adenoid, oral, skin, lung, testes, ovaries, cervix, endometrium, bladder, stomach, epithelium, etc.

In another aspect, a method of treating an individual suffering from a cellular proliferative disorder, particularly cancer, is provided, comprising administering to said individual an effective amount of at least one compound according to Formula I disclosed herein, or a pharmaceutically acceptable salt thereof, either alone, or in combination with a pharmaceutically acceptable carrier.

In yet another aspect, a method of inducing apoptosis of cancer cells, such as tumor cells, in an individual afflicted with cancer is provided, comprising administering to said individual an effective amount of at least one compound according to Formula I, or a pharmaceutically acceptable salt thereof, either alone, or in combination with a pharmaceutically acceptable carrier.

The compounds of Formula I may be administered by any route, including oral, rectal, sublingual, and parenteral administration. Parenteral administration includes, for example, intravenous, intramuscular, intraarterial, intraperitoneal, intranasal, intravaginal, intravesical (e.g., to the bladder), intradermal, transdermal, topical or subcutaneous administration. Also contemplated within the scope of the invention is the instillation of a drug in the body of the patient in a controlled formulation, with systemic or local release of the drug to occur at a later time. For example, the drug may be localized in a depot for controlled release to the circulation, or for release to a local site of tumor growth.

One or more compounds useful in the practice of the present disclosure may be administered simultaneously, by the same or different routes, or at different times during treatment. The compounds may be administered before, along with, or after other medications, including other antiproliferative compounds.

The treatment may be carried out for as long a period as necessary, either in a single, uninterrupted session, or in discrete sessions. The treating physician will know how to increase, decrease, or interrupt treatment based on patient response. The treatment may be carried out for from about four to about sixteen weeks. The treatment schedule may be repeated as required.

In particular, cancer treating compositions may include the DOTAM chelators used in combination with radioisotopes and PSMA peptide targeting moieties to further enhance treatment properties. The radioisotopes, such as 212Pb, 203Pb, 64Cu, and/or other radionuclide α-emitters, have high linear energy transfer (LET) emission and short path lengths that irradiates a short distance, such as within about 1-2 cell diameters, and/or that may not require oxygenation or reproduction to irreversibly damage (e.g., kill) a tumor cell.

As shown herein, these components form stable complexes with isotopes that seek to prevent dissociation of the lead radioisotope from the conjugate under mildly acidic conditions, such as in vivo. Examples herein use 212Pb, 203Pb, or 64Cu as the radioisotope bound to the DOTAM for the targeted imaging and therapy of cancer. Other radioisotopes may include, for example, iron, cobalt, zinc, and other metals with a density of over about 3.5 g/cm3.

The DOTAM-based cancer treating compositions may also form stable complexes with other radioisotopes, and therefore selectively deliver the radioisotopes to the cancer cells and prevent their dissociation that could induce cytotoxic effect in normal cells. Due to their properties, such compositions may be used for treatment of PSMA tumors with specific cancer treatment wherein the isotopes are selectively delivered to the PSMA expressing cancer cells by targeting moieties, such as octreotate, octreotide, or other somatostatin analogs.

The radioisotopes may be used, for example, to provide a source of alpha irradiation via indirect emission. The radioisotopes (e.g., 212Pb, 203Pb, 64Cu, 67Cu, etc.) may be combined with chelators (e.g. DOTAM, TCMC, etc.) and targeting moieties, into a cancer targeting composition for rapid uptake of the composition into the cancer cells. The DOTAM chelators may be used to avoid dissociation of the radioisotope from the conjugate under mildly acidic conditions, such as within the patient's body.

The targeted cancer treatment may involve the use of radioisotopes bound to the chelators which are bound to the targeting moiety which recognizes and binds to cell surface receptors expressed on (or which are up-regulated on) specific cancer cells. This may cause binding of the radioisotope-chelators to the specific cancer cells, and thus targeted radiation of the specific cancer cell when the radioisotope undergoes radioactive decay.

Treatment (e.g., imaging and/or apoptosis) of cancer cells may involve use of emitters (such as e.g., α (alpha), β (beta), γ (gamma), and/or positron emitting radioisotopes) as the radioisotope(s). The α-emitting radioisotopes may be delivered to targeted cancer cells by PSMA targeting moieties, which are known in the art. These α-emitting radioisotopes may be of particular interest because they have a high LET compared to other radioisotopes such as 177Lu, 90Y, and/or other β-emitters, and may deposit their high energy within about a 70 to about a 100 μm long pathway tracking within about 1 to about 2 cancer cell clusters. This high LET radiation may not depend on active cell proliferation or oxygenation, and/or the resulting Deoxyribonucleic acid (DNA) damage caused by α-particles may be more difficult to repair than that caused by β-emitting radioisotopes, due to α-emitting radioisotopes higher LET.

The α-emitting radioisotopes may have an LET that is powerful, and is also generally limited to within the internal region of the cancer cell. The emissions from the α-emitting radioisotopes may also have the ability to cause irreversible damage, such as oxygenation or reproduction, to the cancer cell that does not require waiting for the life cycle of the cancer cell. Further still, α-emitting radioisotopes can cause death and apoptosis of the cancer cells that developed resistance to β-emitter therapy.

The α-emitting radioisotopes may be, for example, produced during decay of lead based radioisotopes, such as 212Pb radioisotopes. The 212Pb is a β-emitting radioisotope with a half-life of about 10.6 hours with a radioactive emission profile having decay products which are α-emitters having the properties of α-emitting radioisotopes. Since 212Pb decays to 212Bi (which is an α-emitting radioisotope having a half-life of about 60 minutes), which decays whether by α-emission to 208Tl (with a half-life of about 3 min), which decays by β-emission to 208Pb (which is stable), or by β-emission to 212Po (with a half-life of about 0.3 μs), which decays by α-emission to 208Pb.

The use of a radioisotope with a relatively long half-life, such as 212Pb having a half-life of about 10.6 hours, may allow for centralized production of radiolabeled compositions at the radiopharmacy and shipment to the clinic where it is administered to the patient. The α-emitter decay of 212Bi may be maximized to occur within the cancer cells, thereby providing maximum alpha radiation damage once inside the cancer cells and their apoptosis and killing of the cancer cell. After α-emission by the 212Bi, the ultimate result is the stable 208Pb.

### Examples

Non-Clinical Study Reports

The nonclinical studies of ⁶⁴Cu-DOTAM-PSMA determined the time-dependent accumulation of this agent in tumor and normal organs. These studies were done in PSMA-overexpressing LNCap and 22Rv1-derived xenografts generated in three different strains of male mice: a) Athymic Nude Mice (Envigo, Indianapolis, Ind. and Taconic, Rensselaer, N.Y.), b) NOG (NOD/Shi-scid/IL-2Rγ^{null}) mice (Taconic, Rensselaer, N.Y.) and c) R2G2 (Rag2-Il2rg Double Knockout) mice (Envigo, Indianapolis, Ind.). All non-clinical studies of ⁶⁴Cu-DOTAM-PSMA were performed by the Drug Discovery and Preclinical Core Facility at RadioMedix, Inc., Headquarter.

### Example 1—PET Imaging of ⁶⁴Cu-DOTAM-PSMA in LNCap and 22Rv1 Derived Xenografts Generated Hi Athymic Nude Mice

Methods

Tumor Inoculation

About 5×10⁶ LNCap and 22Rv1 cells suspended in 100 μL of RPMI 1640 with 50% Matrigel (Corning, Corning, N.Y.) were subcutaneously injected into upper flank of 6-7-week-old mice. Xenografts were generated in Athymic Nude Mice (Envigo, Indianapolis, Ind. and Taconic, Rensselaer, N.Y.). When xenograft tumor reached the size of 0.25 cm³ in diameter, all mice were randomly divided in groups for PET imaging and biodistribution studies.

PET Imaging Methods and Analysis

PET/X-Ray imaging studies were performed using GENISYS⁴ scanner (Sofie Bioscience, Curlver City, Calif.). Mice were anesthetized using with isoflurane (2% in 98% oxygen) and their temperature was kept at 38° C. with a heating lamp during injection of the agent and image acquisition All images were corrected for photon attenuation, but scatter correction was not applied. Maximum-Likehood Expectation Maximization was used to create final images volumes. Static PET scans were acquired approximately at 1 h, 2 h, 4 h after intravenous injection of ⁶⁴Cu-DOTAM-PSMA in 200 μL volume. The image acquisition time was 10 minutes. VivoQuant software (Invicro, Boston, Mass.) was used to determine the ROI (Sum) for tumor, liver, kidney, muscle and salivary gland which were equivalent to % ID/g uptake of agent at various time points.

Results and Conclusions

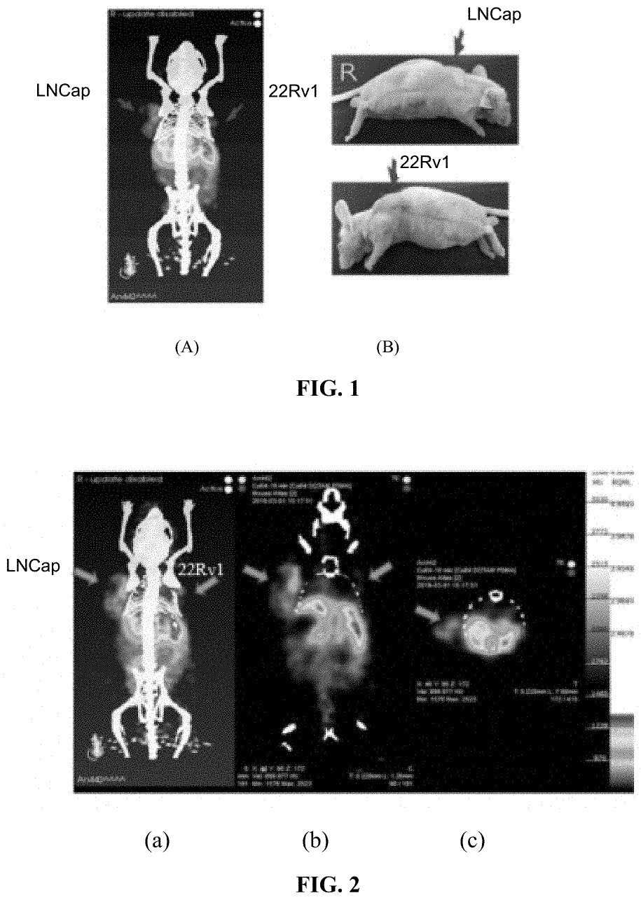

⁶⁴Cu-DOTAM-PSMA PET scans have shown accumulation of agent in tumors derived from both LNCap and 22Rv1 xenografts as early as 1 h post injection. The retention of agent in tumors was followed up to 4 h post injection (FIG. 1). The highest non-target uptake of agent was observed in liver due to the enzymatic trans-chelation of ⁶⁴Cu from ⁶⁴Cu-DOTAM-PSMA by enzymes, Cu/Zn peroxidase dismutase (SOD) and metallothionein. This in vivo trans-chelation of ⁶⁴Cu-DOTA-labeled agents has been already described in literature [a) Anderson C J, Ferdani R. Copper-64 radiopharmaceuticals for PET imaging of cancer: advances in preclinical and clinical research. Cancer Biother Radiopharm. 2009; 24(4):379-393; b) L. A. Bass, M. Wang, M. J. Welch, C. J. Anderson, In Vivo Transchelation of Copper-64 from TETA-Octreotide to Superoxide Dismutase in Rat Liver, Bioconjugate Chem. 20001; 14527-532; c) Miao L, St Clair D K. Regulation of superoxide dismutase genes: implications in disease. Free Radic Biol Med. 2009; 47(4):344-356; d) Ying Wang, Robyn Branicky, Alycia Noë, Siegfried Hekimi, Superoxide dismutases: Dual roles in controlling ROS damage and regulating ROS signaling, JCB, June 2018, 217 (6) 1915-1928]. The expression level and the catalytic activity of Cu/Zn SOD is changed in the physiological states (e.g. aging) and age-associated diseases such as cardiovascular diseases, neurodegenerative diseases, and cancer [Griess B, Tom E, Domann F, Teoh-Fitzgerald M. Extracellular superoxide dismutase and its role in cancer. Free Radic Biol Med. 2017; 112:464-479]. A low expression of SOD correlates with reduced survival of cancer patients suggesting that the loss of extracellular redox regulation promotes cancer progression. The reduction of SOD expression in cancer patients should translate into higher enzymatic stability of ⁶⁴Cu-DOTAM-based conjugates, similarly to results observed during the clinical studies of ⁶⁴Cu-DOTATATE [Johnbeck C B, Knigge U, Loft A, Berthelsen A K, Mortensen J, Oturai P, Langer S W, Elema D R, Kjaer A., Head-to-Head Comparison of ⁶⁴Cu-DOTATATE and ⁶⁸Ga-DOTATOC PET/CT: A Prospective Study of 59 Patients with Neuroendocrine Tumors, J Nucl Med. 2017 March, 58(3):451-457].

FIG. 1 depicts the microPET imaging studies of ⁶⁴Cu-DOTAM-PSMA (injected dose 45 uCi) in LNCap (left flank) and 22Rv1(right flank) xenografts generated in the Athymic Nude Mice. Images were acquired 1 h post-injection. (A) is the reconstructed fused PET/CT scan and (B) are photos of mice showing the actual size of the implanted tumors. The agent is retained in both LNCap and 22Rv1-derived tumors.

The microPET imaging studies acquired at 2 h post-injection confirmed retention of ⁶⁴Cu-DOTAM-PSMA in the LNCap and 22Rv1-derived tumors generated in athymic nude mice (FIG. 2). This result suggests that the enzymatic trans-chelation of ⁶⁴Cu happens during initial distribution of agent through blood stream just after its i.v. injection and that this process has no significant impact on the agent already retained in tumor.

FIG. 2 depicts microPET imaging studies of ⁶⁴Cu-DOTAM-PSMA in LNCap (left flank) and 22Rv1(right flank) xenograft mice done at 2 h post-injection; a) the reconstructed fused PET/CT scan; b) coronal view; c) axial view. The agent is retained in both LNCap and 22Rv1-derived tumors, according to one or more examples of the disclosure.

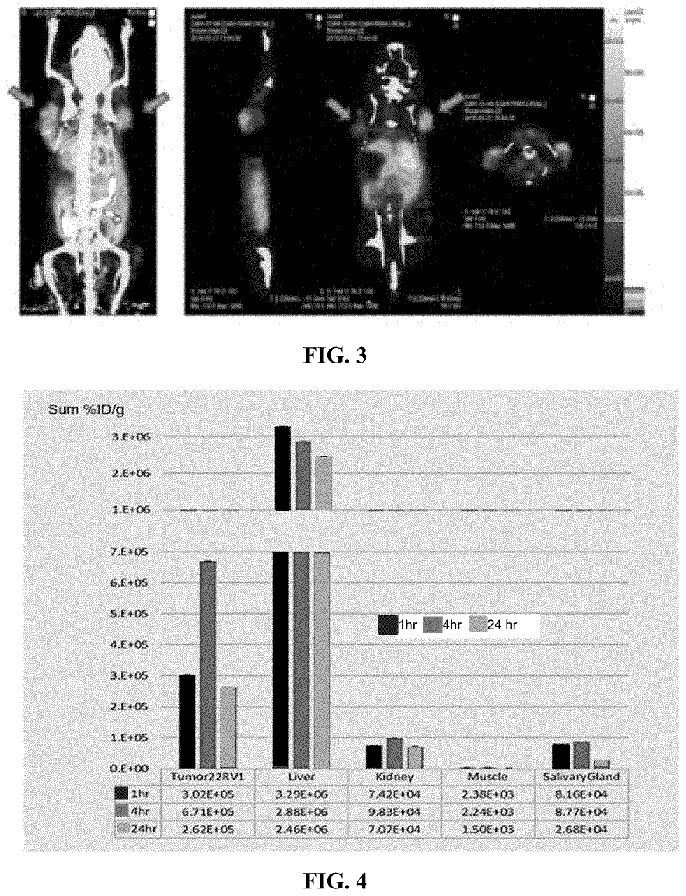

The follow up micoPET imaging studies of ⁶⁴Cu-DOTAM-PSMA done at 4 h post injection confirmed its tumor retention in LNCap and 22Rv1 cancer cells (FIG. 3).

FIG. 3 depicts microPET imaging studies of ⁶⁴Cu-DOTAM-PSMA (62.3 uCi) in LNCap (left flank, volume 500 mm³) and 22Rv1 (right flank, volume 192 mm³) xenografts mice done at 4 h post-injection; a) the reconstructed PET/CT fused scans; b) the sagittal view; c) coronal view; d) axial view. The agent is retained in both LNCap and 22Rv1 tumors as well as non-target organ, liver, according to one or more examples of the disclosure.

Quantitative PET imaging studies of ⁶⁴Cu-DOTAM-PSMA completed in athymic nude mice, allowed to determine the time-dependent differences in the uptake of agent in tumor and normal organs (FIG. 4). ⁶⁴Cu-DOTAM-PSMA accumulation in tumor has reached the highest values of 6.71E+05% ID/g at 4 h post injection. The liver uptake of agent was reduced slightly from 3.3E+06% ID/g at 1 h post injection to 2.5E+06% ID/g at 24 h timepoint. The uptake of ⁶⁴Cu-DOTAM-PSMA in kidneys and salivary glands were comparable at early time points (1 h post injection), however the accumulation of agent in salivary glands was reduced by 2-folds at 24 h while it was remained almost unchanged in kidneys.

FIG. 4 shows graphs plotting the time-dependent changes in distribution of ⁶⁴Cu-DOTAM-PSMA in 22RV1 tumor and normal organs (liver, kidneys, muscle and salivary glands).

### Example 2— PET Imaging of ⁶⁴Cu-DOTAM-PSMA Acquired in the Low Volume LNCap and 22Rv1-Derived Xenografts Generated Athymic Nude Mice (Tumor Volume 0.1-0.150 mm³)

Methods:

Tumor Inoculation

About 5×10⁶ LNCap and 22Rv1 cells suspended in 100 μL of RPMI 1640 with 50% Matrigel (Corning, Corning, N.Y.) were subcutaneously injected into upper flank of 6 week-old Athymic Nude Mice (Envigo, Indianapolis, Ind.). When xenograft tumor reached the size of 0.1 cm³ in diameter, all mice were randomly divided in groups for PET imaging and biodistribution studies.

PET Imaging Methods and Analysis

PET/X-Ray imaging studies were performed using GENISYS⁴ scanner (Sofie Bioscience, Curlver City, Calif.) according to protocol described the Study Report PSMA-001.

### Results and Conclusions

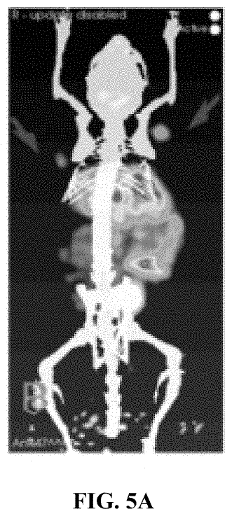

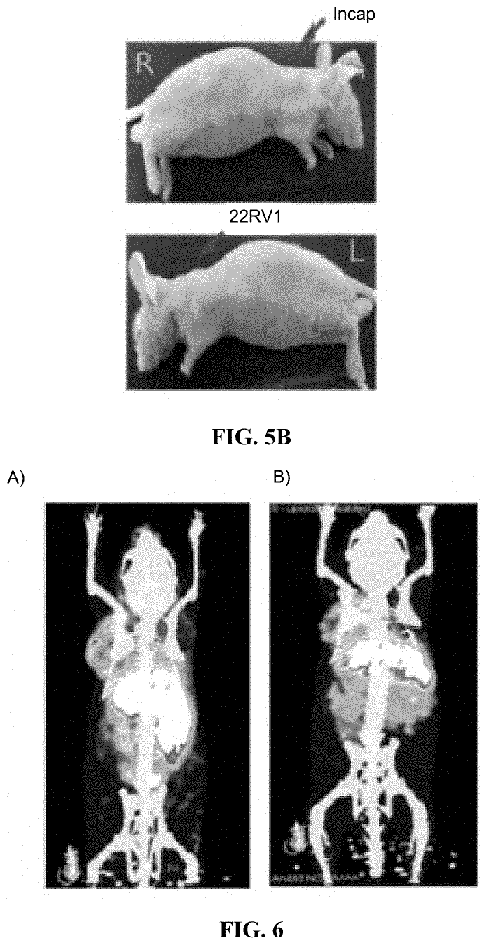

The uptake of ⁶⁴Cu-DOTAM-PSMA in the PSMA-overexpressing tumors does not depend on the tumor volume and the agent can detect tumors smaller than 150 mm³ (FIGS. 5A and 5B).

FIG. 5A depicts microPET imaging studies of ⁶⁴Cu-DOTAM-PSMA in LNCap (left flank) and 22Rv1 (right flank) xenografts generated in the athymic nude mice. The scans were acquired at 1 h post-injection. The tumors volumes were below 150 mm³. FIG. 5B are photos of mice showing size of the implanted tumors, according to one or more examples of the disclosure.

### Example 3— PET Imaging of ⁶⁴Cu-DOTAM-PSMA Acquired at in LNCap and 22Rv1 Xenografts in NOG Strain of Mice (Tumor Volume 0.1-0.150 mm³)

To evaluate the differences in the tumor accumulation and organ distribution of ⁶⁴Cu-DOTAM-PSMA in different strains of mice, the micro PET imaging studies were done in the xenografts generated in NOG mice.

Methods:

Tumor Inoculation

About 5×10⁶ LNCap and 22Rv1 cells suspended in 100 μL of RPMI 1640 with 50% Matrigel (Corning, Corning, N.Y.) were subcutaneously injected into upper flank of 6-7-week-old NOG (NOD/Shi-scid/IL-2Rγ^{null}) mice (Taconic, Rensselaer, N.Y.). When xenograft tumor reached the size of 0.25 cm³ in diameter, all mice were randomly divided in groups for PET imaging and biodistribution studies.

PET Imaging Methods and Analysis

PET/X-Ray imaging studies were performed using GENISYS⁴ scanner (Sofie Bioscience, Curlver City, Calif.) according to protocol described the Study Report PSMA-001.

### Results and Conclusions

The accumulation and retention of ⁶⁴Cu-DOTAM-PSMA in LNCap tumor generated in NOG mice was similar to the one observed in athymic nude mice. There was slightly higher uptake of agent in kidneys and bladder at 1 h post injection (FIG. 6).

FIG. 6 depicts microPET imaging studies of ⁶⁴Cu-DOTAM-PSMA in LNCap xenografts generated in NOG mice; Studies were done at 1 h (A) and 24 h (B) post-injection.

### Example 4— the Biodistribution Studies of ⁶⁴Cu-DOTAM-PSMA Done at in LNCap and 22Rv1-Derived Xenografts in Athymic Nude Mice

Methods

Mice bearing LNCap and 22Rv1 xenografts were injected via the tail vein with 50-100 μCi of ⁶⁴Cu-DOTAM-PSMA reconstituted in 150-200 μL of saline. At 1 h, 2 h, and 24 hrs. post injection, while under anesthesia blood was collected by cardiac puncture and mice were sacrificed by cervical dislocation. The heart, lung liver, stomach, pancreas, spleen fat, kidney, muscle, intestines, skin and tumor were collected. Each organ was weighed, and the tissue radioactivity was measured with an automated gamma counter (2470 Wizard2 Gamma Counter, Perkin-Elmer, Waltham, Mass.). The percentage of injected dose per gram of tissue (% ID/g) was calculated. All measurements were corrected for decay.

### Results and Conclusions

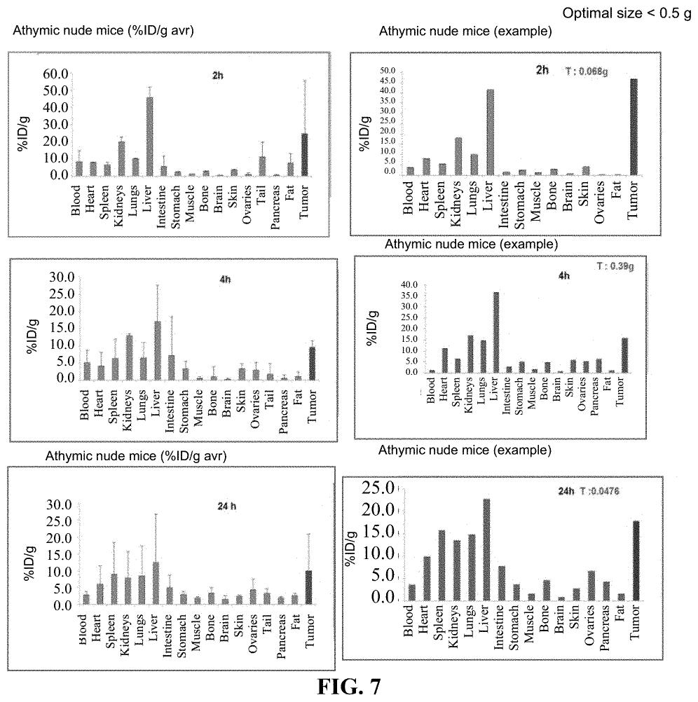

Tumor uptake of ⁶⁴Cu-DOTAM-PSMA was in the wide range of 24.8±31.1% ID/g at 2 h post injection and decreased to 9.7±10.9% ID/g at 4 h (FIG. 7). The off-target accumulation of drug in the liver and kidneys measured at 2 h post-injection was 45.8±6.2% ID/g, 20.0±2.9% ID/g, respectively. The accumulation of agent in liver was further reduced to 17.1±10.1 ID/g and its renal retention to 13.0±0.6% ID/g, at 4 h timepoint. As it was mentioned before, the high liver uptake of agent can be explained by the trans-chelation of ⁶⁴Cu from DOTAM conjugate in the reaction catalyzed by peroxidase dismutase. The renal retention of ⁶⁴Cu-DOTAM-PSMA can be correlated with expression of PSMA receptors in proximal tubules in kidneys. These of off-target uptake of agent should not affect the diagnostic properties of ⁶⁴Cu-DOTAM-PSMA.

Retention of ⁶⁴Cu-DOTAM-PSMA in tumor has not changed significantly at 24 h post injection (10.0±11.1% ID/g) compared to 4 h time point. The uptake of agent in liver and kidneys at 24 h timepoint decreased to 12.4±11.9% ID/g, and 7.8±7.9% ID/g, respectively.

FIG. 7 shows graphs plotting the biodistribution studies of ⁶⁴Cu-DOTAM-PSMA in athymic nude mice done at 1 h, 2 h and 24 h post-injection. The liver and kidneys are the off-target organs showing the highest accumulation of agents.

### Example 5— the Biodistribution Studies of ⁶⁴Cu-DOTAM-PSMA Done at in LNCap and 22Rv1-Derived Xenografts Generated in R2G2 Strain of Mice

Methods

R2G2 strains of mice bearing LNCap and 22Rv1 xenografts were injected via the tail vein with 50-100 μCi of ⁶⁴Cu-DOTAM-PSMA reconstituted in 150-200 μL of saline. At 1 h, 2 h, and 24 hrs. post injection, while under anesthesia blood was collected by cardiac puncture and mice were sacrificed by cervical dislocation. The heart, lung liver, stomach, pancreas, spleen fat, kidney, muscle, intestines, skin and tumor were collected. Each organ was weighed, and the tissue radioactivity was measured with an automated gamma counter (2470 Wizard2 Gamma Counter, Perkin-Elmer, Waltham, Mass.). The percentage of injected dose per gram of tissue (% ID/g) was calculated. All measurements were corrected for decay.

### Results and Conclusions

⁶⁴Cu-DOTAM-PSMA has shown very similar accumulation rate in tumors (LNCap and 22Rv1) generated in R2G2 strain and NOG strain of mice at 2 h post injection. The liver retention of trans-chelated Cu64 was higher in R2G2 mice strain compared to NOG strain but it was still lower the one observed in athymic nude at the same timepoint. The tumor retention of ⁶⁴Cu-DOTAM-PSMA measured at 24 h post injection was much more favorable in R2G2 strain than NOG strain. The higher rate of trans-chelation of ⁶⁴Cu observed in NOG mice could contribute to lower uptake of agent in tumor and its significantly higher uptake in liver at 24 h time point.

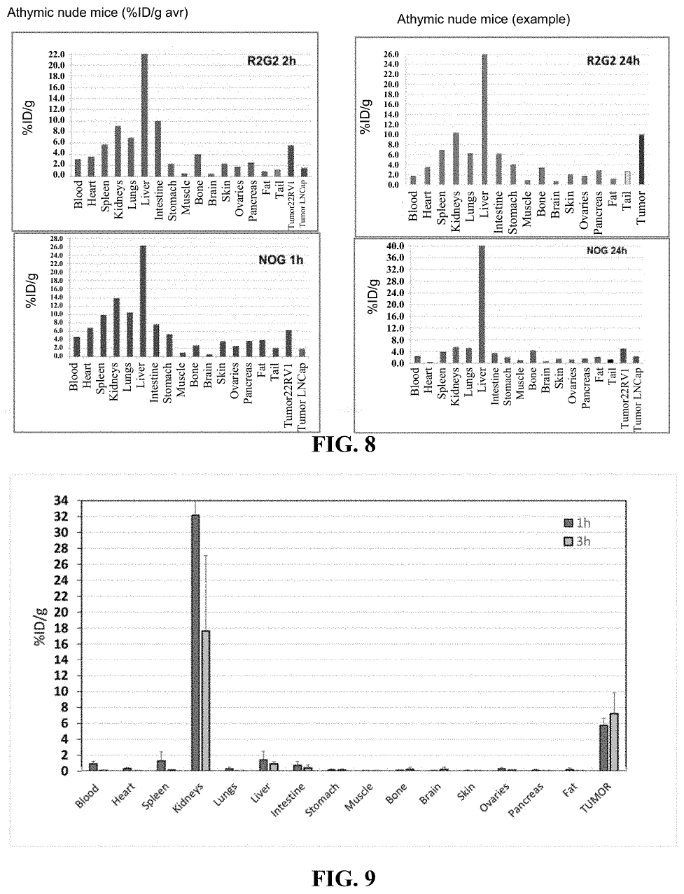

FIG. 8 shows graphs plotting the biodistribution studies of ⁶⁴Cu-DOTAM-PSMA in LNCap and 22RV1 xenografts of R2G2 mice done at 2 h and 24 h post-injection and of NOG mice done at 1 h and 24 h post-injection.

Justification for not Requiring Single Dose Toxicity Studies

The pre-clinical studies disclosed herein confirmed in vivo selectivity and specificity of ⁶⁴Cu-DOTAM-PSMA toward PSMA-positive LNCap and 22Rv1-based xenografts. The kidney retention of ⁶⁴Cu-DOTAM-PSMA is similar to the one observed for other radiolabeled PSMA derivatives and it correlates with expression of PSMA receptors in the proximal tubules. The high liver uptake of drug observed in animal models is due to the trans-chelation of Cu64 by peroxidase dismutase. Since, the expression and/or activity of this enzyme is decreased in cancer patients, the in vivo release of ⁶⁴Cu from conjugate should be also reduced.

The amount of DOTAM-PSMA to be administered per patient will not exceed the micro dosing amount of 100 μg, and it will be well below the known toxicity for PSMA or the chelate DOTAM used in Phase 1 clinical trial (NCT01384253) and exploratory clinical studies (IND #130960). All these results suggest that no toxicity studies are needed for the microdosing PET imaging studies during eIND clinical studies of ⁶⁴Cu-DOTAM-PSMA.

### Example 6—Biodistribution Studies of ²¹²Pb-DOTAM-PSMA in LNCap and Derived Xenografts Generated in Athymic Nude Mice

Methods

Athymic strains of mice bearing LNCap xenografts were injected via the tail vein with 15 μCi of ²¹²Pb-DOTAM-PSMA reconstituted in 150-200 μL of saline. At 1 h, 3 h, post injection, while under anesthesia blood was collected by cardiac puncture and mice were sacrificed by cervical dislocation. The heart, lung liver, stomach, pancreas, spleen fat, kidney, muscle, intestines, skin and tumor were collected. Each organ was weighed, and the tissue radioactivity was measured with an automated gamma counter (2470 Wizard2 Gamma Counter, Perkin-Elmer, Waltham, Mass.). The percentage of injected dose per gram of tissue (% ID/g) was calculated. AH measurements were corrected for decay.

### Results and Conclusions

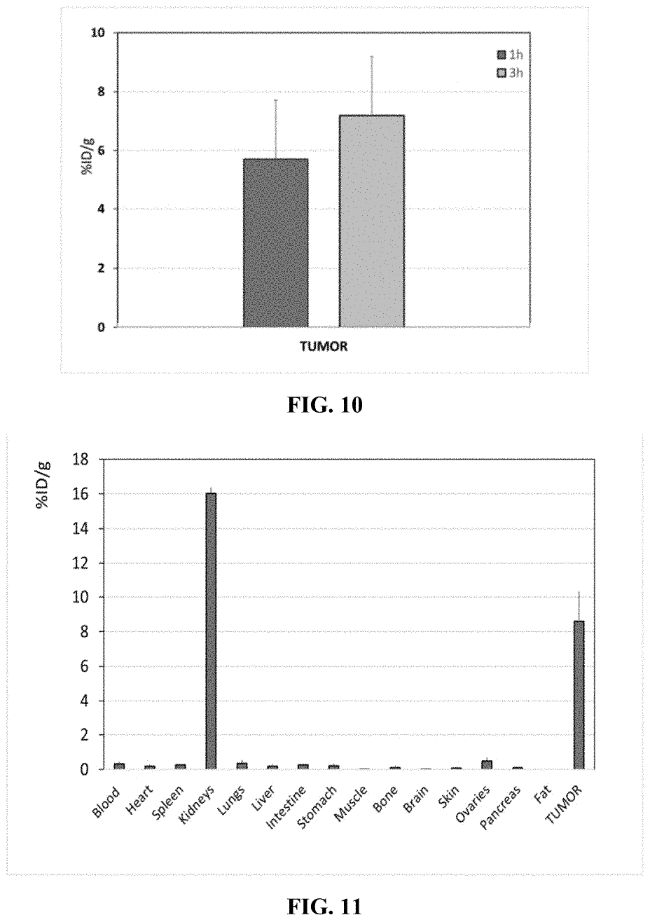

Tumor uptake of ²¹²Pb-DOTAM-PSMA was in the range of 5.7±0.9% ID/g at 1 h post injection and increased to 7.2±2.6% ID/g at 3 h (FIG. 9). The agent was eliminated from the blood stream through kidneys and its renal retention was 32.2±15.6% ID/g at 1 h post-injection and decreased 55% to 17.7±9.4% ID/g at 3 h time point. The renal retention of ²¹²Pb-DOTAM-PSMA can be correlated with expression of PSMA receptors in proximal tubules in kidneys. There was not uptake of agent in bone and spleen that confirmed the high in vivo stability of ²¹²Pb-DOTAM-PSMA complex. The side by side comparison of accumulation of ²¹²Pb-DOTAM-PSMA in LNCAP xenografts at 1 h and 3 h post-injection is shown in FIG. 10.

### Example 7— Biodistribution Studies of ²⁰³Pb-DOTAM-PSMA in LNCap and Derived Xenografts Generated in Athymic Nude Mice

To determine the effect of chelating on the organ distribution of DOTAM-PSMA, the initial comparative biodistribution studies were done using ²⁰³Pb-DOTAM-PSMA. The ²⁰³Pb is a gamma emitter (279 keV) with t1/2=51.9 h, suitable for single-photon emission computed tomography (SPECT) imaging. The ²⁰³Pb is an ideal surrogate for ²¹²Pb α-particle therapy because both isotopes share identical chemical properties.

Methods

Athymic strains of mice bearing LNCap xenografts were injected via the tail vein with 40 μCi of ²⁰³Pb-DOTAM-PSMA reconstituted in 200-250 μL of saline. At 1 h and 3 h post injection, while under anesthesia blood was collected by cardiac puncture and mice were sacrificed by cervical dislocation. The heart, lung liver, stomach, pancreas, spleen fat, kidney, muscle, intestines, skin and tumor were collected. Each organ was weighed, and the tissue radioactivity was measured with an automated gamma counter (2470 Wizard2 Gamma Counter, Perkin-Elmer, Waltham, Mass.). The percentage of injected dose per gram of tissue (% ID/g) was calculated. All measurements were corrected for decay.

### Results and Conclusions

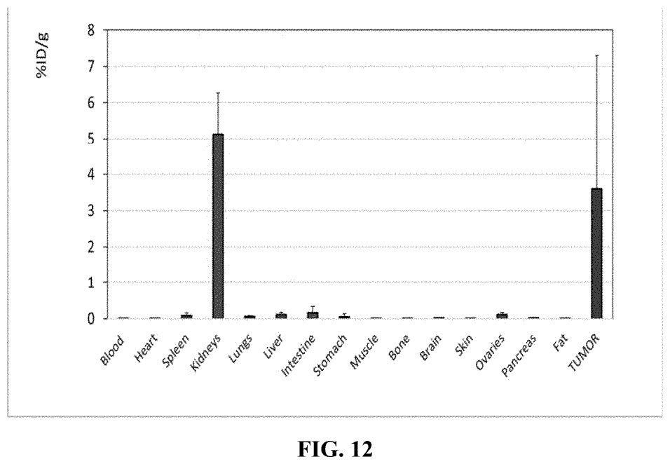

Both ²⁰³Pb-DOTAM-PSMA and ²¹²Pb-DOTAM-PSMA have shown very similar normal organs distribution. The high renal retention of both agents correlates with expression of PSMA receptor in kidneys and can be also attributed to positive +2 charge of these conjugates. Tumor uptake of ²⁰³Pb-DOTAM-PSMA was in the range of 16.1±0.8% ID/g at 1 h post injection (FIG. 11). There was no uptake of agent in normal organs such as bone and spleen.

²⁰³Pb-DOTAM-PSMA was retained in tumor at 3 h time post-injection and the uptake was higher than 4.8% ID/g. The renal retention of ²⁰³Pb-DOTAM-PSMA decreased 32% compared to earlier time points with no additional uptake of agent in any other normal organs. FIG. 12 represents the biodistribution studies of ²⁰³Pb-DOTAM-PSMA in PSMA-overexpressing xenografts of athymic nude mice done at 3 h post-injection.

### Example 8— Radiochemical Stability of Pb203-RMX-PSMA

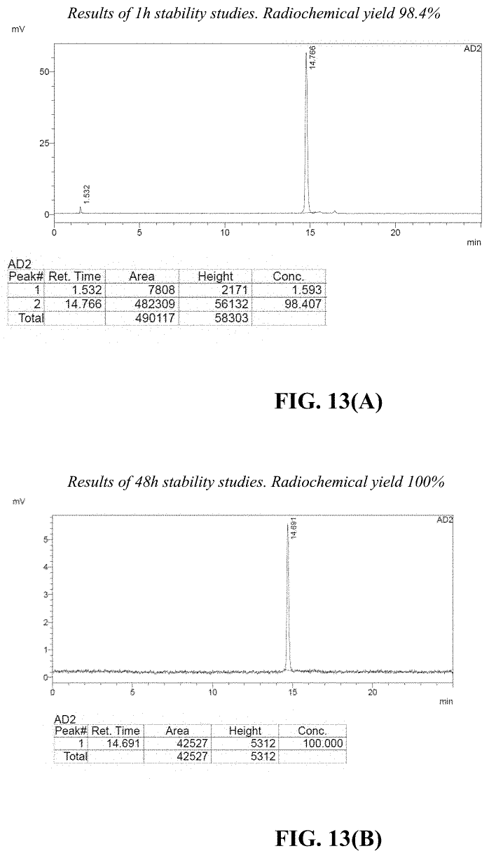

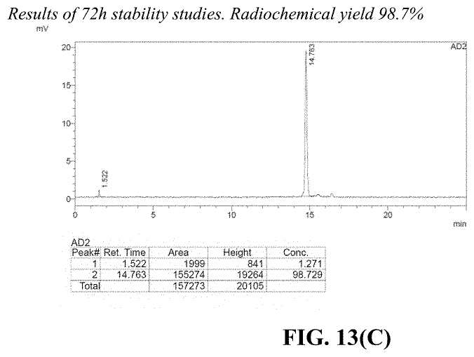

For testing radiochemical stability, RMX-PSMA (5 μg) in 0.4 M NH4OAC (400 μl) was radiolabeled the with 15 mCi (30 μl, 0.1 HCl). The reaction was completed after 10 min. incubation at room temperature and the aliquots (200 ul) were left at room temperature for up 72 hours. Samples were analyzed by radio/UV HPLC (Shimadzu) without additional dilutions. Selected chromatograms are shown in FIG. 13A-C. The radiochemical yield of Pb203-RMX-PSMA synthesis was higher than 98% and radiotracer was stable up 72 hours at room temperature.

As indicated by the experimental data provided herein, a combination of certain radioisotopes chelated using DOTAM or TCMC conjugated to PSMA receptor targeting moieties provides treatment properties, such as increased radiochemical stability, enhanced binding and increased uptake by cancer cells, and/or high LET emission within cancer cells that results in their apoptosis and/or targeted biodistribution.

The methods herein may be performed in any order and repeated as desired.

While the embodiments are described with reference to various implementations and exploitations, it will be understood that these embodiments are illustrative and that the scope of the inventive subject matter is not limited to them. Many variations, modifications, additions and improvements are possible. For example, various combinations of part or all of the techniques described herein may be performed.

Plural instances may be provided for components, operations or structures described herein as a single instance. In general, structures and functionality presented as separate components in the exemplary configurations may be implemented as a combined structure or component. Similarly, structures and functionality presented as a single component may be implemented as separate components. These and other variations, modifications, additions, and improvements may fall within the scope of the inventive subject matter.

Insofar as the description above and the accompanying drawings disclose any additional subject matter that is not within the scope of the claim(s) herein, the inventions are not dedicated to the public and the right to file one or more applications to claim such additional invention is reserved. Although a very narrow claim may be presented herein, it should be recognized the scope of this invention is much broader than presented by the claim(s). Broader claims may be submitted in an application that claims the benefit of priority from this application.

What is claimed is:

1. A cancer targeting compound for treatment of cancer cells overexpressing PSMA comprising a radioisotope, a chelator, and a PSMA-targeting moiety, wherein the PSMA-targeting moiety is linked to the chelator.

2. The compound of claim 1 wherein the chelator comprises a nitrogen ring structure.

3. The compound of claim 2 wherein the nitrogen ring structure comprises DOTAM.

4. The compound of claim 1, wherein the radioisotope is selected from the group consisting of ⁶⁴Cu, ⁶⁷Cu, ²⁰³Pb, and ²¹²Pb.

5. The compound of claim 1, wherein the PSMA-targeting moiety comprises a PSMA receptor targeting peptide.

6. A composition for diagnosing cancer cells overexpressing PSMA comprising the compound of claim 1.

7. A composition for treating cancer cells overexpressing PSMA comprising the compound of claim 1.

8. The compound of claim 1, wherein the compound has the following structure 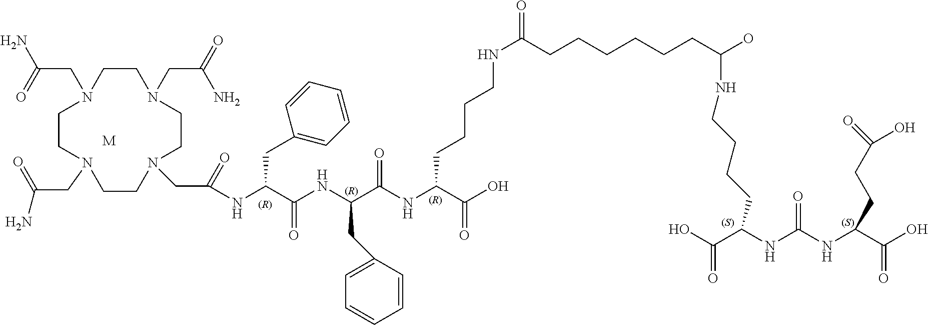
and wherein M is a radioisotope.

9. The compound of claim 8, wherein the radioisotope is selected from the group consisting of ⁶⁴Cu, ⁶⁷Cu, ²⁰³Pb, and ²¹²Pb.

10. The compound of claim 8, wherein the radioisotope is ⁶⁴Cu.

11. The compound of claim 8, wherein the radioisotope is ⁶⁷Cu.

12. The compound of claim 8, wherein the radioisotope is ²⁰³Pb.

13. The compound of claim 8, wherein the radioisotope is ²¹²Pb.

14. A kit for diagnosing cancer cells overexpressing PSMA comprising a radioisotope, a chelator, and a targeting moiety.
